(19) United States
(12) Patent Application Publication
(10) Pub. No.: US 2024/0325681 A1
(43) Pub. Date: Oct. 3, 2024

(54) CANNULA AND METHOD FOR PRODUCING A CANNULA

(71) Applicant: Xenios AG, Heilbronn (DE)

(72) Inventors: Patrick Rauh, Freiburg (DE); Sven Filipon, Heilbronn (DE); Uwe Fabritius, Brackenheim (DE); Thomas Bingener, Stuttgart (GM); Patrick Wörthwein, Cleebronn (DE)

(21) Appl. No.: 18/042,727

(22) PCT Filed: Aug. 18, 2021
(86) PCT No.: PCT/EP2021/072934

§ 371 Date: Feb. 23, 2023

(30) Foreign Application Priority Data
Aug. 24, 2020 (DE) 10 2020 005 161.8

(51) Int. Cl. A61M 25/00

(52) U.S. Cl. CPC A61M 25/0009; A61M 2207/10

(57) ABSTRACT

The present invention relates to methods for manufacturing a cannula as well as cannulas, in particular for conveying body fluids, and corresponding circulatory support systems, which in particular reduce disadvantages which may occur during the manufacture and use of the cannulas. Accordingly, a method is suggested according to which a hollow body (10) is provided which comprises a continuous channel (22) in a longitudinal direction of the hollow body (10) and between opposite ends of the hollow body (10), and wherein the hollow body (10) is slid onto a holding mandrel (12), such that the holding mandrel (12) is at least partially received in the channel (22) and the hollow body (10) at least partially surrounds the holding mandrel (12), wherein the holding mandrel (12) has a predetermined configuration with a substantially circular cross-section. At least one liquid material layer (16, 24) is applied to a predetermined portion (14) of the holding mandrel (12) under rotation and axial displacement of the holding mandrel (12), wherein the predetermined portion (14) is adjacent to one end of the hollow body (10) and is not surrounded by the hollow body (10). The holding mandrel (12) is then immersed or dipped in a liquid cover layer material (18), such that a cover layer (18) extends over the at least one material layer (16; 24) and at least one end region (20) of the adjacent hollow body (10) being adjacent thereto.

### TECHNICAL FIELD

The present invention relates to methods for manufacturing cannulas, in particular for conveying body fluids, as well as cannulas and corresponding circulatory support systems.

### TECHNOLOGICAL BACKGROUND

Cannulas can be inserted into the body of a patient for various medical applications to aspirate or remove body fluids and/or to deliver or administer therapeutic fluids to the patient. For example, cannulas may be adapted for use in the gastrointestinal tract or the digestive tract. They can be used to remove excess and/or physiologically damaging fluids from the body or to administer drugs and/or nutrients locally. Furthermore, cannulas are used in particular for circulatory support. Low-oxygen and carbon dioxide-saturated blood is taken from the patient via a cannula and oxygen-rich and low-carbon dioxide blood can be returned via the same or another cannula.

For fluid delivery, cannulas comprise a hollow body defining a continuous channel, which extends longitudinally and can be inserted into the patient. At one end of the hollow body, a connector and a coupling portion are provided to enable the connection of medical devices such as syringes or components of medical equipment such as pumps.

In prior art cannulas, such connectors and/or coupling portions are either directly cast with a hollow body or produced by one or more dipping or immersion steps of one end of the hollow body in a liquid material. Alternatively, such connectors and/or coupling portions may be manufactured separately from the manufacture of the hollow body, for example, by dipping or casting, and may then be bonded to the hollow body to provide a finished cannula.

### SUMMARY OF THE INVENTION

Based on the known prior art, it is an object of the present invention to improve the production of cannulas and in particular to simplify or shorten the production or manufacturing process.

According to the invention, it was recognized that the production of cannulas or components of a cannula by immersion or dipping according to the prior art involves comparatively long process times.

On the one hand, these are caused by the required number of dipping or immersion processes, which have to be considered for a predefined dimensioning of the cannula. On the other hand, curing times are always required between the dipping or immersion processes. In this manner, a holding mandrel, which can be used for manufacturing, is occupied for a longer period of time for the production of a single cannula and thus not available for the production of further cannulas. In order to produce a larger number of cannulas, a correspondingly large number of holding mandrels must be provided in accordance with the prior art, which makes the entire production process even more expensive. Finally, the at least partially manual execution or operation or support of the method steps are further cost drivers for conventional production processes.

Furthermore, it was recognized in accordance with the invention that due to the shape of the components being used and to be connected and the execution or operation of the method steps according to the prior art, edges or steps are formed on the outer surface of the cannula. Such steps are unfavorable for medical use. They not only make it difficult to insert the cannula into a blood vessel or other body cavities, but can also mechanically damage a patient's anatomical structures or impair the flow profiles of body fluids in the vicinity of the cannula. Furthermore, such irregularities in the cannula wall can impair the mechanical properties, especially since they increase the risk of kinking.

Accordingly, there is a need to simplify and accelerate the manufacturing of cannulas, taking into account functionally relevant parameters for use in a patient undergoing treatment. It is advantageous to avoid costly working steps.

This object is achieved by the independent claims. Preferred embodiments are defined by the dependent claims, the description, and the Figures.

Accordingly, a method for manufacturing a cannula is suggested, which includes the following steps:
providing a hollow body, which comprises a continuous channel in a longitudinal direction of the hollow body and between opposite ends of the hollow body;
sliding the hollow body onto a holding mandrel, such that the holding mandrel is at least partially received in the channel and the hollow body at least partially surrounds the holding mandrel, the holding mandrel having a predetermined configuration with a substantially circular cross-section;
applying at least one liquid material layer to a predetermined portion of the holding mandrel under rotation and axial displacement of the holding mandrel, wherein the predetermined portion is adjacent to one end of the hollow body and is not surrounded by the hollow body; and
immersing the holding mandrel in a liquid cover layer material such that a cover layer extends over the at least one material layer and at least one end region of the adjacent hollow body adjacent thereto.

The hollow body can be formed as a tubular body and preferably be made of a flexible material, which comprises an inner cavity defined by the channel with terminal openings. Accordingly, the channel provides an inner lumen, which allows a fluidic connection to the outside at opposite ends of the hollow body and is adapted for conveying body fluids. Due to the elongated shape of the hollow body and the channel, the longitudinal direction of the hollow body essentially corresponds to the axial direction of the hollow body and the cannula.

The predetermined portion of the holding mandrel, to which the at least one material layer is applied, forms a free portion of the holding mandrel, when the hollow body or its channel with terminal openings is slid or placed or positioned on the holding mandrel, preferably when the hollow body is completely pushed or slid on the holding mandrel. Due to the design of the holding mandrel, also known as mandrel, the holding mandrel can be dimensioned for a predetermined or predefined length of the hollow body, such that the predetermined free portion or section is directly connected or adjacent to one end of the hollow body. Nevertheless, it can also be provided that the hollow body is only partially pushed or slid onto the holding mandrel and that the hollow body is delimited in the predetermined portion by the holding mandrel or the predetermined portion to provide the desired positioning.

The predetermined portion, preferably the free portion, corresponds preferably in the longitudinal direction to a desired or predetermined length for a connector of the cannula. For example, a ratio of the length of the predetermined portion in the longitudinal direction to the length of the hollow body or the portion of the hollow body surrounding the holding mandrel may be 1:1 to 1:7.

The application of at least one material layer occurs typically successively, preferably continuously, such that at least the first material layer can be applied in a spiral or helical shape by rotation and axial displacement. The material is applied in such a way that the predetermined portion is completely covered by the at least one material layer. Alternatively, however, the material can also be applied completely along the longitudinal direction and a rotation can be carried out successively such that the material layer is applied in layers and/or meandering in the circumferential direction.

Furthermore, the axial displacement can also be achieved by a relative displacement to an application element, wherein either the application element or the holding mandrel is moved axially to provide an axial displacement during application.

Preferably, no movement of the applied material layer takes place during rotation and displacement in order to prevent leakage or an unintentional or uneven distribution of the material during the application process. For example, the application can thus be based on the application speed, the rotational speed, the axial displacement, and/or a state of the material, such as a predefined higher viscosity liquid. This application step is preferably controlled or regulated by a control device.

After application of the at least one material layer and application of the cover layer, the manufactured cannula can be removed directly from the holding mandrel. A surface of the at least one material layer that comes into contact with the holding mandrel during the manufacturing of the cannula defines an inner wall whose shape is determined by the shape of the predetermined portion of the holding mandrel. A fluidic connection with the channel of the hollow body is thus enabled.

In this way, a method for manufacturing a cannula is provided which allows the number of immersion or dipping processes to be considerably reduced. As a result, fewer manual steps are required. Not only are the manufacturing costs and the amount of work reduced, but also an at least partially or essentially automated execution or operation is made possible. According to the invention, the required curing time is shortened accordingly, so that the method can be carried out faster and the existing holding mandrels can be used more efficiently for further manufacturing processes and can be made available again more quickly for use in the manufacture of further cannulas. Thus, the procurement of a larger number of cost-driving holding mandrels can be reduced to a minimum number according to the method according to the invention.

A further significant advantage of this method is the medically advantageous design of the manufactured cannula. This is because the application of at least one material layer or base layer, respectively, ensures that a continuous surface is created in the border area of the applied material layer and the hollow body, which is essentially free of edges, borders and/or protrusions. This means that during the subsequent dipping process, a cover layer can be applied, which in turn is uniformly continuous and does not form a step with the hollow body. This eliminates the disadvantages of conventional processes. A material layer defines a connector and is simultaneously applied to or attached to the hollow body.

A step in the outer surface can not only make it more difficult for the treating physician to insert the cannula into body cavities. It can also cause damage to surrounding anatomical structures, such as the vascular endothelial layer, during insertion. If the cannula is inserted into a patient's bloodstream, a step may also cause undesirable flow phenomena, such as turbulence and a correspondingly unfavorable (blood) flow behavior. This can impair the pumping or conveying of body fluids, such as blood, or even locally increase the tendency to clot. According to the invention, the application of the at least one material layer or base layer, respectively, and the subsequent application of the cover layer largely avoids such a step, so that the insertion and placement of the cannula can be facilitated and patient safety can be appropriately taken into account. Furthermore, irregularities in the cannula wall, which would impair the mechanical properties, can be effectively avoided, so that the risk of kinking is accordingly reduced.

It is preferable to apply the at least one material layer in such a way that it is flush with an end face of the adjacent end of the hollow body so as to provide a flush transition from the hollow body to the connector without a step or edge. Typically, the at least one material layer is applied in such a way that no gap or void is created between the material layer and the adjacent end of the hollow body and the material layer is evenly or uniformly and completely distributed over the entire end face or the surface at the end of the hollow body, which extends in the radial direction and may be substantially formed by the thickness of the wall of the hollow body. Due to the uniform and complete distribution, elevations of the material layer with respect to the thickness or height of the hollow body at the end of the hollow body or at its end face are kept low or are avoided, so that the at least one material layer is advantageously aligned flush with the circumference of the hollow body in this end region. The distribution can be further supported, for example, by suitable selection of the application speed, the rotational speed and/or the viscosity of the material.

Accordingly, the at least one material layer is preferably applied evenly or uniformly and with a substantially continuous thickness. In this way, it is possible to avoid any undesirable elevations that might affect the uniform design of the cover layer to the greatest possible extent, not only on the end face, but also along the entire longitudinal direction and on the circumference of the at least one material layer. As a result, a cover layer may be formed without steps and elevations and in a correspondingly uniform manner.

Furthermore, the holding mandrel can be dipped or immersed in the liquid cover layer material in such a way that the cover layer is flush with the circumference of the end region of the hollow body and/or the circumference of a portion of the hollow body leading away from the end region. In this way, a continuous and, with regard to the portion of the hollow body to which no cover layer is applied, continuous cover layer is applied. The thickness of the cover layer can be predefined, for example, by suitable selection of the type and/or speed of dipping and by the viscosity of the cover layer material. It can be essentially continuous or increase in thickness in the longitudinal direction and away from the adjacent end of the hollow body.

The end region can be dimensioned in such a way that a ratio of the length of the end region in the longitudinal direction of the hollow body to the length of the at least one material layer is about 1:2 to about 1:10. Preferably, this ratio is about 1:4. The cover layer can be used both to connect the at least one material layer to the hollow body and to facilitate the insertion of the cannula into a patient, such that the ratio can be selected appropriately, for example, based on a predetermined structural stability and/or suitability for an anatomical dimensioning.

In order to achieve different properties of the at least one material layer and the cover layer, the material of the cover layer may differ from the material of the at least one material layer. For example, the cover layer may be made of a material with a relatively higher surface roughness for improved handling or preferably have a lower surface roughness for insertion into a body cavity. Likewise, the material of the cover layer may have a higher relative elasticity or be more flexible than the at least one material layer, making it easier to grasp the cannula when inserting it. Alternatively, for certain applications, the cover layer material may have a higher relative stiffness to provide improved structural stability. Such a design may be advantageous with respect to any potential coupling portions that are attached to the end of the cover layer opposite the adjacent end of the hollow body. The material of the cover layer and the at least one material layer may include, for example, thermoplastic, solvent-based materials such as polyurethanes or silicones and/or the material may have a Shore hardness of between 40A and 80D.

Although the above-mentioned advantages of the method may generally be achieved with one material layer, it is also possible to provide at least one additional material layer, i.e. a total of at least two material layers. Thus, after the application of a first liquid material layer and before the application of the cover layer, at least a second liquid material layer can be applied over the first material layer under rotation and axial displacement of the holding mandrel. The application of at least two material layers may have the advantage that the method can cover a larger range of variants, e.g. with regard to diameter or thickness of the finished product. For example, a single material layer may be limited to a certain minimum or maximum thickness or height or radial extension of the material due to a given or predetermined viscosity of the material and/or a preset application speed, in order to ensure uniform distribution. This also allows the first material layer to be applied as a base layer, for example, which is independent of the diameter or wall thickness of the hollow body and accordingly has a lower height. By applying at least two material layers, preferably two layers of material, fine tuning can be performed and a larger radial extension can be achieved.

Furthermore, by applying at least two material layers, different properties can be taken into account. For example, a first material layer can be formed in particular from a material designed for improved biocompatibility and for pumping or conveying body fluids, while the second material layer can be designed or adapted, for example, with properties for improved structural stability and/or improved bonding to the cover layer.

Accordingly, at least two liquid material layers may be applied, preferably two material layers, wherein at least two of these material layers can comprise the same material or also different materials. Examples of such materials include thermoplastic, solvent-based materials as described in the above.

Depending on the type of material used in the at least one material layer and/or the cover layer, at least partial curing of the at least one material layer may be required. Preferably, the at least one liquid material layer is cured prior to the application of the cover layer, wherein curing is preferably performed under the influence of heat and/or light. The curing may have been completed or be only partially completed before the application of the cover layer, depending on the type of material bond between the at least one material layer and the cover layer to be achieved.

For example, the at least one material layer may have a predetermined viscosity and/or surface tension, which may be advantageous for bonding with the cover layer, but which advantageously does not cause an uneven or non-uniform distribution of the material layer upon immersion or dipping. Thus, partial curing of the at least one material layer may be preferred if, for example, improved wetting, bonding and/or cross-linking is to be achieved by the cover layer. Alternatively, the at least one material layer may be fully cured before the cover layer is applied. For example, an even or uniform and continuous surface of the at least one material layer, which may be advantageous for the even or uniform application of the cover layer, may potentially only be achieved after complete curing of the at least one material layer.

Preferably, the curing is carried out after the application of the respective material layer and/or during the application of the respective material layer. For example, curing can be carried out or accelerated by supplying heat, wherein the heat is provided by heating the holding mandrel or by heating the air surrounding the holding mandrel. Accordingly, curing can be triggered or effected during application and/or after application. When applying the respective material layer, an individually adjusted heat setting is preferred in order to ensure a further improved distribution of the material during the application process for given material characteristics, e.g. viscosity. By adding additional heat, a curing or hardening of the material layer can subsequently be achieved, if necessary locally.

Preferably, the curing of the at least one material layer after application of the respective material layer is effected by heated ambient air, for example, at a temperature between 20° C. and 30° C. or about 25° C. and/or for a period of about two to 10 hours, preferably five to eight hours or about seven hours. The curing can be performed in a particularly preferred way by rolling, wherein the holding mandrel is rotated or rolled accordingly. In this way it is possible to prevent irregularities due to drop formation or leakage of the material, especially in the case of materials with a lower viscosity.

In order to facilitate the application of the at least one material layer, this step can be carried out with the assistance of a lathe. In this way, the holding mandrel can be held by the lathe at least at one end and the hollow body can be pushed or slid on at the opposite end of the holding mandrel up to the predetermined portion. The use of the lathe allows a fast and at least partially automated, preferably fully automated application. This considerably reduces the number of manual steps required in the manufacturing of the cannula. Furthermore, a controlled application can be carried out in this way, such that a more even or uniform distribution can be achieved. Axial displacement can be achieved, as described in the above, by a relative displacement to an application element, wherein either the application element or the holding mandrel, or both if necessary, are moved axially to allow axial displacement during application. For example, the lathe may include an application element and be configured to axially displace an accommodated holding mandrel relative to the application element.

Preferably, an application element in the form of a nozzle is provided on or in the lathe. The application can be carried out by means of a nozzle, which can be displaced in axial direction relative to the holding mandrel. The nozzle can optionally be heated in order to take into account a material-specific viscosity suitable for the application step. The material is applied by means of the nozzle while rotating the holding mandrel, wherein the nozzle should move axially and relative to the already applied material, after one or more rotations of the holding mandrel. The material can be applied in a spiral shape to the end face or the adjacent end of the hollow body. The speed of rotation, however, allows the respective turns to flow or diffuse, so that the material layer forms a uniform and continuous layer in both the axial and rotational directions.

The thickness of the at least one material layer can be determined by the speed of the lathe, the feed rate of the nozzle and the flow rate of the nozzle. Thus, the rotational speed and the feed rate can be the same for each application and for different cannulas or hollow bodies and corresponding wall thicknesses only the feed rate can be variable. Alternatively, the rotational speed, the feed rate and the delivery or flow rate can be variable. Preferably, the speed is between 150 and 210 rpm, the feed rate is between 70 and 120 mm/min and/or the delivery or flow rate is between 0.3 and 3 g/min.

To further automate the application step and to avoid the occurrence of possible application errors as far as possible, a relative axial displacement between the nozzle and the holding mandrel can be sensor-driven. Within its detection range, the sensor can preferably detect a rotation of the holding mandrel, the presence of at least one material layer on the holding mandrel, a thickness of the at least one material layer, at least the end of the end region of the hollow body, and/or a thickness or height of the hollow body at the end of the hollow body. In this way it can be ensured, for example, that a first material layer is applied evenly (or uniformly) and, if necessary, a second material layer is applied evenly (or uniformly) to the first material layer. The application of the material can be performed, for example, on the basis of a height or thickness of the material on the holding mandrel, which is determined by the thickness of the first material layer and is detected by a sensor.

By detecting the adjacent end of the hollow body, an automatic guidance of the nozzle over the total length of the at least one material layer can be provided, even if different holding mandrels are used. Thus, the predetermined portion can have different lengths for different holding mandrels, such that an axial displacement of the nozzle does not have to be restricted by the lathe or a predefined axial limitation of the nozzle. The detection of the end of the hollow body, however, renders it possible to apply the material automatically even in such cases. Furthermore, the detection of a thickness or height of the hollow body at the end of the mounted hollow body enables the application to be realized for different heights or thicknesses of the hollow body. Preferably, a thickness and/or height of the applied material is detected in order to automatically determine a number and/or quantity or thickness, respectively, of the required material layers. Furthermore, the delivery rate of the material can be controlled by a pump like a micro gear pump, wherein the delivery rate of the material can be adjusted according to the viscosity and desired layer thickness by means of the pump setting.

The coating can be done by dip coating. Thus, the cover layer can be easily applied to the at least one material layer and the end region of the hollow body. Preferably, a viscosity as well as a speed of dipping or immersion can determine a thickness and a course of the thickness of the cover layer.

Preferably, at least one further dipping process is carried out after dipping, wherein the cover layer material is cured after each dipping process or step. Accordingly, at least two cover layers may be provided. The number of dipping processes is preferably 5 to 20, in particular 8 to 14. Between each dipping process, the cover layer material is cured, for example at a temperature between 20° C. and 40° C., for example 30° C., and/or for a duration of one hour to three hours. The multiple dipping processes allow the wall to be successively strengthened, however, without creating potentially unfavorable irregularities on the circumference of the cannula.

The immersion or dipping depth can be further reduced for each successive dipping procedure, preferably by about 1 mm to about 3 mm. In this way, the cannula is given an essentially continuous or steplessly increasing wall thickness at the end opposite the hollow body, which allows further elements to be advantageously attached at this end.

The holding mandrel may be held by a linear press fit during dipping and preferably also during curing, preferably by a prism. This can considerably reduce swinging of the workpiece carrier, especially in comparison to punctual clamping, such that an improved stability and an even or uniform distribution of the cover layer during dipping and also during curing is provided.

Due to the application of the at least one material layer according to the invention, which preferably is performed automatically and with simultaneous curing for further acceleration of the method, only one dipping step may be necessary to provide an even or uniform and continuous connection between the hollow body and the at least one material layer. The required application volume of the cover layer can be further reduced considerably by the application of the at least one material layer according to the invention, since only the at least one material layer and the end region of the hollow body are coated with the cover layer. In addition, the selective coating also considerably reduces the total curing time, even in the case of one or more successive dipping processes. The manufacturing time of the cannula can thus be considerably reduced by combining the application of the at least one material layer and the dipping or immersion, while in continuous manufacturing operation a larger number of holding mandrels are available for the production of additional cannulas than in prior art processes. This in turn reduces the number of holding mandrels to be used compared to conventional processes.

In order to accelerate the manufacturing process in other ways, the hollow body is preferably produced by extrusion. In this way, a large number of hollow bodies can be produced in a simple manner and separately from the application of the at least one layer of material, i.e. the hollow bodies can be provided in particular before the process according to the invention is carried out. This enables an essentially continuous and cost-effective process, wherein high quantities can be provided with relatively short production times. The separate production of the hollow body does not cause any irregularities on the outer surface or on the circumference of the manufactured cannula. This is because the application of the at least one material layer to the predetermined portion of the holding mandrel creates a continuous surface. A cover layer applied to it can be formed continuously and without steps, unlike in conventional prior art processes. In contrast to this, in conventional processes a connector is directly cast with a hollow body or produced by one or more dipping processes.

The hollow body and/or the channel with its lumen can have an essentially continuous circumference, cross-section and/or diameter. Accordingly, the hollow body, as described in the above, can for example be tubular and essentially formed as a tube wall. The continuous circumference and/or cross-section enables a substantially equal dimensioning of the cannula in longitudinal direction, which facilitates the insertion into the body cavity and provides that flow profiles in the inserted state do not differ substantially in the longitudinal direction of the cannula.

The channel can thus have an essentially continuous cross-section and/or diameter, so that body fluids can be conveyed correspondingly evenly or uniformly. Any unfavorable pressure differences are largely eliminated. Alternatively, for certain applications, the hollow body can be formed asymmetrical, ellipsoidal and/or curved, at least in sections or portions. Furthermore, one end of the hollow body, to which no cover layer is applied, can be designed as a tip and preferably as a rounded and/or atraumatic tip, which facilitates the insertion of the cannula into the body of a patient.

A uniform flow behavior along the longitudinal direction of the entire cannula can also be achieved by the choice of the material of the at least one material layer. This can be achieved by creating a surface that touches the holding mandrel during the manufacture of the cannula, for example, with a predetermined surface roughness and/or surface tension. For example, the surface roughness can be predetermined by a surface treatment of the holding mandrel, wherein the outer surface of the holding mandrel was preferably treated by glass bead blasting. Furthermore, the hollow body and the at least one material layer preferably consist of the same material or at least of similar materials or at least of materials with essentially similar surface properties. By manufacturing using the same material, the method is naturally further simplified, with the aim of reducing the manufacturing costs or process costs.

A structural stability of the cannula can be defined by the design or structure of the hollow body. Preferably, the hollow body is at least sectionally wire-reinforced, preferably essentially over its entire length. The wire reinforcement, which can be arranged in a longitudinal direction, e.g. spirally, can be embedded in the material of the hollow body and can be arranged in such a way that the material neither protrudes from nor enters the circumference of the hollow body or the channel, respectively. For example, the wire reinforcement can be provided by coextrusion. On the one hand, the wire reinforcement facilitates insertion into the body cavity and ensures improved patient safety. On the other hand, it can also prevent the finished product from buckling or kinking during an operation on the patient. Even if the product is bent, it can hence be ensured that the channel is not completely blocked in any case.

In order to fluidically connect the cannula or hollow body to a medical device, a connection portion or region may also be required that is adapted to hold or receive a predetermined amount of fluids. For example, a hollow chamber may be provided to provide pressure equalization and/or a mixing reservoir for blood to be discharged or supplied. Accordingly, the cross-section of the predetermined portion of the holding mandrel adjacent to the hollow body can increase in size as it leads away from the hollow body.

Thus, by applying the at least one material layer, a connection or connector may be created, wherein the connection increases in size starting from the hollow body, preferably continuously (i.e. without steps). The outer surface of the connector in longitudinal section then advantageously exhibits a continuous, stepless shape. The shape can be essentially straight, so that a radial extension increases evenly over the length of the connector. However, it can also be rounded, wherein an increase in radial extension is not continuous, at least in sections, and the shape may accordingly be parabolic.

Preferably, the portion of the holding mandrel adjacent to the hollow body is conical or tapered in shape. Such a shape has the advantage that this end region of the cannula is easy to grasp. This also provides an improved or more advantageous flow profile.

During the execution or operation of the above mentioned method steps, the hollow body is held or fixed by the holding mandrel in order to prevent a relative displacement in axial direction and a relative rotation to the holding mandrel. Preferably, the hollow body is held by the holding mandrel during the application of the at least one material layer and the application of the cover layer by means of a press fit. For example, at the end of the holding mandrel or the hollow body to which no material layer or cover layer is applied, a protrusion may be provided, which preferably can be selectively extended or folded out or pushed out and/or extends in radial direction. If the hollow body is made of a partially elastic material, the projection may even be provided permanently or protrude from the holding mandrel. When the hollow body is pushed or slid onto the holding mandrel, the hollow body can be deformed at this point and be held by the protrusion.

The hollow body may be pushed or slid completely onto the holding mandrel so that the protrusion or projection is axially adjacent to the end of the hollow body and axially restricts the hollow body at this end when the hollow body has been placed or slid on. The press fit can also be supported by the predetermined portion or free portion, for example by being conical or similarly shaped, thus providing an axial limitation at the opposite end, when the hollow body is pushed or slid on.

Preferably, a press fit is provided, either alternatively or additionally, by a radially inward force exerted by the hollow body. For example, the hollow body may be elastic and the cross-section of the opening of the hollow body may be smaller than the circumference of the holding mandrel. The hollow body can first be inflated or otherwise enlarged to increase the diameter of the opening and then be pushed or slid onto the holding mandrel. The hollow body can then be restored to its original state. Accordingly, the hollow body can also be clamped around the holding mandrel.

For various medical uses or applications, it may also be advantageous for the cannula to have a predetermined coupling, such as a Luer-Lock based coupling. Accordingly, the at least one first material layer can provide a connector of the cannula, wherein a coupling part is attached to an end of the connector opposite the hollow body, preferably by adhesive bonding. For example, the coupling part may have one or more inlets for fluidic connection to a syringe and/or medical device such as a circulatory support system.

Furthermore, the cannula can be further coated at least in sections or portions after the cover layer has been applied, e.g. to increase the biocompatibility of the cannula. This additional (surface) coating can have the form of a “nanolayer”, for example. In particular, it can change or preferably reduce an existing surface tension, improve wetting and/or include one or more drugs or physiologically active components, such as an anticoagulant or blood thinner, which is/are released successively as a retard formulation.

The above object is further solved by a cannula, wherein the cannula is preferably formed or adapted to deliver body fluids or therapeutic fluids. The cannula comprises a hollow body, which comprises a continuous channel in a longitudinal direction of the hollow body and between opposite ends of the hollow body, and a connector, which comprises at least one material layer that is flush with an end face of one end of the hollow body, wherein the connector also comprises a continuous channel, which is fluidically connected to the channel of the hollow body. Furthermore, the cannula comprises a cover layer, which is arranged over the at least one material layer and extends over an adjacent end region of the hollow body. The cover layer is preferably flush with the circumference of the end region of the hollow body and/or the circumference of a section of the hollow body leading away from the end region.

As described in the above, the material layer provided according to the invention has the advantage, among other things, that a flush or aligned connection is provided between the cover layer arranged thereon and the hollow body, which is not realized in conventional prior art cannulas. In conventional cannulas either a step is present or the cannula is completely manufactured by dipping or immersin processes. While a step, as described above, is disadvantageous and also makes the insertion of the cannula more difficult for a user, cannulas made in one piece have the disadvantage that they are made of one material. This can result in biocompatibility problems, insertion problems and/or problems with flow conditions. In addition, individual molds are required for each cannula to be produced, the provision of which is expensive, wherein the number of pieces is limited to the number of molds during production. In addition, cannulas which are manufactured completely by dipping have the considerable disadvantage that the production is very time-consuming. These disadvantages can be considerably reduced or even avoided by the presence of at least one material layer.

The cannula and especially the hollow body of the cannula can be essentially tubular and preferably have a continuous circular cross-section in longitudinal direction. The channel can be surrounded by or defined by a tube wall. In a design as an alternative to a continuous circular embodiment, the hollow body can be at least partially asymmetrical, ellipsoidal and/or curved. At the end of the hollow body opposite the connector, a tip can typically be provided, preferably a rounded and/or atraumatic tip, which facilitates the insertion of the cannula into the body of a patient.

The above advantages and preferred embodiments with respect to the method according to the invention also apply to the cannula according to the invention and vice versa, where applicable.

Accordingly, a ratio of the length of the end region in the longitudinal direction of the hollow body to the length of the at least one material layer can be 1:2 to 1:10, preferably about 1:4.

At least two material layers may also be provided, wherein a second material layer is arranged between a first material layer and the cover layer, wherein the first and second material layers are formed from the same material or different materials. Alternatively, or additionally, the cover layer and the at least one material layer can be formed from different materials. The hollow body and the at least one material layer are preferably made of the same material.

Preferably, the hollow body and/or the channel of the hollow body have a circumference, cross section and/or diameter that remains essentially constant over its length. Furthermore, the hollow body can be wire-reinforced at least in sections or portions, preferably over its entire length.

The connector preferably has a circular cross-section in the longitudinal direction. The cross-section is particularly preferred to be concentric to the channel of the hollow body or to the circumference of the hollow body, if the hollow body also has a substantially circular cross-section. Preferably, the cross-section of the connector increases in the direction away from the hollow body, such that the connector is preferably conical or tapered. The wall thickness of the connector can be essentially constant in longitudinal direction. Alternatively, or additionally, the thickness of the cover layer can also increase in a direction away from the end region of the hollow body.

A coupling part can also be attached to the end of the connector opposite the hollow body. In this way, the cannula can be used for a variety of applications, for example, by providing access by the coupling part for the removal or delivery of body fluids such as blood. For example, the coupling part can be used to fluidically connect the cannula to one or more syringes and/or a circulatory support system.

Accordingly, the cannula is preferably adapted to convey (in the form of a withdrawal or supply or return) body fluids. Its use in the circulatory support of a patient is preferred. The suitability of the cannula for insertion into the carotid or jugular vein or alternatively into the inguinal vein and/or vena cava of a patient is also preferred.

The cannula is preferably manufactured according to the method according to the invention. In this way, the at least one material layer and the cover layer are adapted to each other in such a way that optimum wetting and a continuous, stepless outer surface of the cannula are provided. At the same time, improved structural stability is also ensured.

The above object is further achieved by a circulatory support system, which includes a cannula according to the invention. The circulatory support system may include at least one blood pump, which may be coupled, for example, to an outlet at a proximal end of the cannula or to a connector or coupling part of the cannula, and by means of which blood may be withdrawn from the patient's cardiovascular system and conveyed to a gas exchanger, such as a membrane oxygenator, and subsequently returned to the patient. For the supply of oxygen-enriched and low-carbon dioxide blood, an additional cannula can be optionally provided, which is fluidically connected to the extracorporeal circulation. The circulatory support system can therefore also preferably include a membrane oxygenator.

Accordingly, the cannula can also be used to support the circulation of a patient.

### BRIEF DESCRIPTION OF THE DRAWINGS

Preferred further embodiments of the invention are presented in more detail in the following description of the Figures, in which:

FIG. 1 is a schematic representation of the method steps for the manufacture of a cannula according to the invention;

FIG. 2 is a schematic longitudinal section of a cannula according to the invention;

FIG. 3 is a schematic longitudinal section of an alternative embodiment of a cannula according to the invention; and

FIGS. 4A to 4C are schematic representations of the cannula after a different number of dipping processes.

### DETAILED DESCRIPTION OF PREFERRED EMBODIMENTS

In the following, preferred embodiments will be explained in more detail with reference to the accompanying Figures. In the Figures, corresponding, similar, or like elements are denoted by identical reference numerals and repeated description thereof may be omitted in order to avoid redundancies.

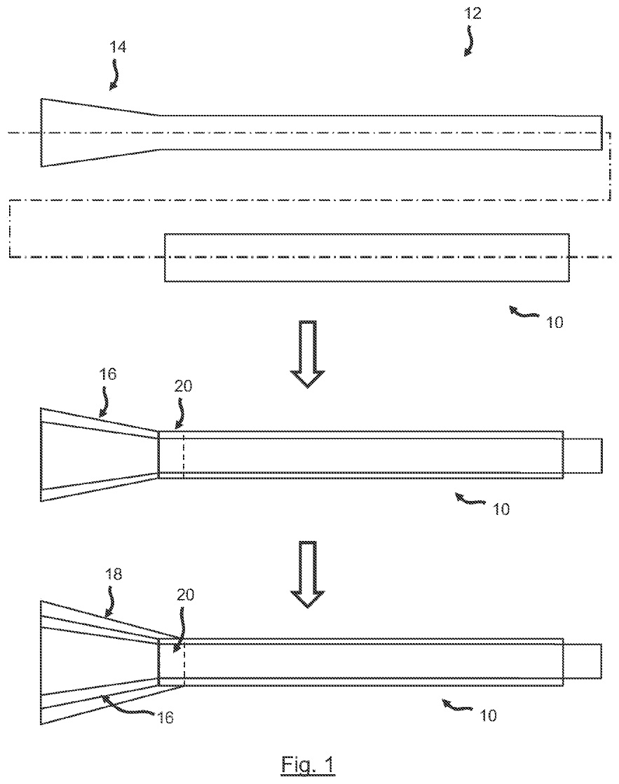

FIG. 1 shows a schematic representation of the respective process steps of the method for manufacturing a cannula according to the invention. First, a hollow body 10 is provided, which is manufactured, for example, by extrusion and has an essentially constant cross-section in a longitudinal direction, which is circular in shape according to the present example. The hollow body 10 comprises a continuous channel (not shown), which extends in the longitudinal direction of the hollow body 10 from one end to the opposite end of the hollow body 10. This enables the conveying of liquids, such as body fluids or therapeutic solutions, from one end to the other end of the hollow body 10. The hollow body 10 in this case is tubular. It thus comprises a continuous tube wall, which has an essentially continuous wall thickness and defines the continuous channel. The tube wall can be wire-reinforced at least in sections, wherein one or more wires are preferably embedded in the wall material of the hollow body 10 in a spiral form.

The hollow body 10 is pushed, slid or placed on a holding mandrel 12, wherein the hollow body 10 is preferably made of an elastic material to facilitate the sliding or placing. Furthermore, sliding on can be facilitated by radial expansion of the hollow body 10 or its channel, e.g. by inflating the hollow body 10. The sliding is performed in such a way that the hollow body 10 or its channel surrounds the holding mandrel 12 in a direction as indicated by the dotted line. In this way, the holding mandrel 12 is at least partially accommodated in the channel.

After being pushed or slid on, the hollow body 10 is returned to its original state so that a radially inwardly acting force is provided, since the cross-section of the opening of the hollow body 10 is smaller than the circumference of the holding mandrel 12 in the present example. At one end of the hollow body 10, the hollow body 10 is delimited by a predetermined portion 14 of the holding mandrel 12, which is conically shaped or formed and defines the region for the application of at least one first material layer 16. In this way, the hollow body 10 is held by the holding mandrel 12 by means of a press fit in the mounted or pushed on state. Further process steps, which require a movement of the holding mandrel 12, thus do not cause any relative movement between the hollow body 10 and the holding mandrel 12.

Then, while rotating the holding mandrel 12, a first material layer 16 is applied to the predetermined potion 14 in the axial direction that is defined by the longitudinal direction of the holding mandrel 12 or the hollow body 10, so that a first material layer is applied to and arranged on the holding mandrel 12. The application can be performed spirally and typically in such a way that the holding mandrel 12 is completely covered by the first material layer 16 in the predetermined portion 14. The first material layer 16 is aligned or flush with one end face as well as with the circumference of the adjacent end region 20 of the hollow body 10. Thus, the first material layer 16 and the hollow body 10 form a continuous and stepless surface on the outer surface, which is free of elevations, grooves and edges.

During the application of the first material layer 16 and/or after the application of the first material layer 16, the first material layer 16 cures, e.g. by applying heat by heating the holding mandrel 12. The holding mandrel 12 is then dipped in a cover layer material 18, so that a cover layer 18 extends over the entire surface of the first material layer 16 and an end region 20 of the hollow body 10. The end region 20 forms a region or portion that is provided for improved mechanical connection between the hollow body 10 and the first material layer 16 that forms a connector and allows a transition to the cover layer 18 and a connector of the cannula. A ratio of the length of the end region 20 in the longitudinal direction of the hollow body 10 to the length of the first material layer 16 can be about 1:2 to about 1:10 and is about 1:4 in the present example. A cover layer 18 is provided on the applied first material layer 16, which is flush with the end region 20 of the hollow body and is continuously and steplessly formed on the outer surface or circumference and does not include any elevations such as projections, grooves or edges.

After the first material layer 16 has been completely applied and the cover layer 18 has been applied, the hollow body 10 including the cover layer 18 and the connector, which is defined by the first material layer 16, can be removed from the holding mandrel 12, if necessary after the first material layer 16 and/or the cover layer 18 has cured. In this way, a cannula with a continuous channel is provided. Adhesion of the first material layer to the portion of the holding mandrel is advantageously avoided, e.g. by curing the material in such a way that no adhesion occurs. To improve the surface properties, the cannula can also be coated. For certain applications, appropriate couplings or connectors may furthermore be required, which can be attached to the end of the connector opposite the hollow body 10.

A corresponding cannula is shown in a longitudinal section in FIG. 2. This longitudinal section shows the advantageous structure of the first material layer 16 and the cover layer 18. On the other hand, it can be seen that the first material layer 16 defines a channel of the connector 26, which is aligned and/or flush with channel 22 of the hollow body 10 and thus forms a continuous channel, which can be used, for example, to convey therapeutic solutions in liquid form or body fluids such as blood.

An alternative embodiment of the cannula is shown in FIG. 3 in a corresponding longitudinal section. In this design, in addition to the first material layer 16, a second material layer 24 is provided, which has been applied over the first material layer 16 after the application of the first liquid material layer 16 and before the application of the cover layer 18, while rotating and axially shifting the holding mandrel 12. The first and second material layers 16, 24 can be made of the same or different materials.

As described in the above, different diameters or thicknesses of the hollow body 10 can be set by applying at least two material layers 16, 24. Specific properties of the product can be provided by suitable selection of the materials. For example, the first material layer 16 may be formed in particular from a material designed for improved biocompatibility and for conveying body fluids, while the second material layer 24 may be adapted, for example, with properties for improved structural stability and/or reinforced bonding with the cover layer 18. According to the present example, the cover layer 18 is made of a different material than the first material layer 16 and the second material layer 24.

The first and second material layers 16, 24 can also be made of the material of hollow body 10 or the embedding material of hollow body 10. Accordingly, by applying the first and second material layers 16, 24, a physical boundary line can be minimized or, depending on the material used, even avoided. This can further improve the structural stability of the cannula and the connection between the connector 26 and the hollow body 10.

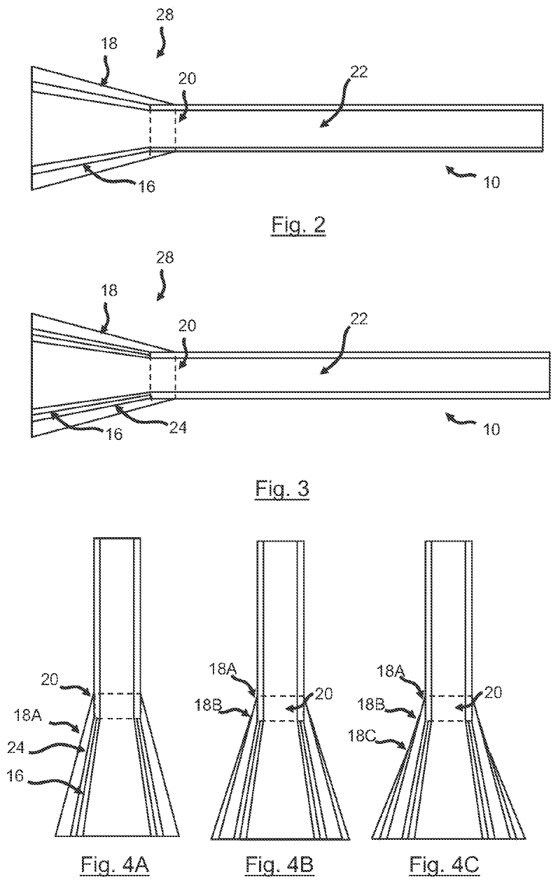

FIGS. 4A to 4C show the cannula (only partially shown) after a varying number of dipping processes. The holding mandrel was immersed or dipped in a cover layer material to provide a first cover layer 18A and, as shown in FIG. 4A, up to the end region 20 of the hollow body so that the cover layer material extends over the end region 20 and the second material layer 24 arranged on top of the first material layer 16. Although only a portion of the hollow body and mandrel are shown, an upper end of the mandrel can be held by a linear interference or press fit during the dipping and the curing to reduce vibrations on a carrier.

After curing the first cover layer 18A, the holding mandrel was again dipped in the cover layer material at a shallower depth, as shown by the arrow, to create a second cover layer 18B, which in this case does not (no longer) extend beyond the end region 20 as shown in FIG. 4B. This process was repeated to provide a third cover layer 18C as shown in FIG. 4C. Accordingly, the dipping processes provide a cover layer whose wall thickness increases substantially continuously to the end opposite the hollow body to provide improved mechanical stability at that end.

Where applicable, all the individual features depicted in the exemplary embodiments may be combined and/or exchanged without leaving the scope of the invention.

### LIST OF REFERENCE NUMERALS

10 Hollow body
12 Holding mandrel
14 Predetermined portion
16 First material layer
18 Cover layer material or cover layer
18A-18C Cover layer material or cover layer
20 End region
22 Channel
24 Second material layer
26 Connector

1. A method of manufacturing a cannula comprising the steps of:
providing a hollow body (10) comprising a continuous channel (22) in a longitudinal direction of the hollow body (10) and between opposing ends of the hollow body (10);
sliding the hollow body (10) onto a holding mandrel (12) such that the holding mandrel (12) is at least partially received by the channel (22) and the hollow body (10) at least partially surrounds the holding mandrel (12), wherein the holding mandrel (12) has a predetermined configuration with a preferably substantially circular cross-section;
applying at least one liquid material layer (16, 24) to a predetermined portion (14) of the holding mandrel (12) under rotation and axial displacement of the holding mandrel (12), wherein the predetermined portion (14) is adjacent to one end of the hollow body (10) without being surrounded by the hollow body (10); and
immersing the holding mandrel (12) in a liquid cover layer material (18) such that a cover layer (18) extends over the at least one material layer (16; 24) and at least an end region (20) of the adjacent hollow body (10) being adjacent thereto.

2.-44. (canceled)
